(19) United States
(12) Patent Application Publication
(10) Pub. No.: US 2025/0333475 A1
(43) Pub. Date: Oct. 30, 2025

(54) CD44 GLYCOEPITOPES AND CHIMERIC VACCINE GLYCOCONJUGATES FOR CANCER THERAPY AND SYNTHESIS METHODS THEREOF

(71) Applicants: I3S - INSTITUTO DE INVESTIGAÇÃO E INOVAÇÃO EM SAÚDE, ASSOCIAÇÃO, Porto (PT); INSTITUTO PORTUGUÊS DE ONCOLOGIA DO PORTO FRANCISCO GENTIL, EPE, Porto (PT); REQUIMTE - REDE DE QUÍMICA E DE TECNOLOGIA ASSOCIAÇÃO, Porto (PT)

(72) Inventors: José Alexandre RIBEIRO DE CASTRO FERREIRA, Porto (PT); André MOREIRA NETO DA SILVA, Porto (PT); Rui Filipe NEVES FREITAS, Porto (PT); Lúcio José DE LARA SANTOS, Porto (PT); Marta Filipa RELVAS DOS SANTOS, Porto (PT); Cristiana MILHAZES GAITEIRO, Porto (PT); Andreia Filipa FERREIRA PEIXOTO, Porto (PT); Andreia Rafaela LINHARES MIRANDA, Carapeços (PT); Dylan GOMES FERREIRA, Vale (PT); Flávia Raquel TEIXEIRA DE CASTRO, Guimarães (PT); Bruno Filipe CARMELINO CARDOSO SARMENTO, Porto (PT); Maria José CARDOSO OLIVEIRA, Porto (PT)

(73) Assignees: I3S - INSTITUTO DE INVESTIGAÇÃO E INOVAÇÃO EM SAÚDE, ASSOCIAÇÃO, Porto (PT); INSTITUTO PORTUGUÊS DE ONCOLOGIA DO PORTO FRANCISCO GENTIL, EPE, Porto (PT); REQUIMTE - REDE DE QUÍMICA E DE TECNOLOGIA ASSOCIAÇÃO, Porto (PT)

(21) Appl. No.: 18/688,999

(22) PCT Filed: Sep. 5, 2022
(86) PCT No.: PCT/PT2022/050025

§ 371 Date: Mar. 4, 2024

(30) Foreign Application Priority Data
Sep. 4, 2021 (PT) 117449
Sep. 2, 2022 (PT) 118181

(51) Int. Cl. C07K 14/705; A61K 39/00; C07K 1/107; C07K 1/22; C12N 9/10

(52) U.S. Cl. CPC C07K 14/70585; C07K 1/1077; C07K 1/22; C12N 9/1051; A61K 39/00; A61K 2039/55572; A61K 2039/70; C12Y 204/01041

(57) ABSTRACT

Glycopeptides derived from short CD44 isoforms lacking amino acids encoded by exons 6-14; presenting one or multiple serine or threonine residues substituted with Tn (GalNAcα-O-Ser/Thr) and/or sialyl-Tn (STn; Neu5Acα2-6GalNAcα-O-Ser/Thr) antigens. Synthesizing the glycopeptides, including one-pot glycosylation of synthetic short isoform CD44 peptides through combination with nucleotide sugars and glycosyltransferases and purification of CD44s-Tn glycopeptides. Immunogenic chimeras derived from the CD44-Tn and/or STn glycopeptides, linked, in polyvalent form, to a carrier immunogenic protein, e.g., KLH CRM197. Conjugating the synthesized CD44s-Tn glycopeptides to the immunogenic protein carriers CRM197 and KLH, generating chimeric glycopeptides, termed CRM197-CD44s-Tn and KLH-CD44s-Tn. CD44-Tn/STn glycopeptides or compositions thereof for treating cancer and pre-neoplastic diseases, e.g., neoplastic diseases expressing short CD44 isoforms, through generating antibodies against cancer cells and treating/preventing cancer by vaccination. The glycopeptides, compositions, synthesis methods and uses can be employed in treating cancer, alone or in combination with immune checkpoint inhibitor therapy, chemotherapy, and radiotherapy.

### CROSS REFERENCE TO RELATED APPLICATIONS

This application is a National Stage of International Application No. PCT/PT2022/050025 filed Sep. 5, 2022, claiming priority based on Portuguese Patent Application No. 117449 filed Sep. 4, 2021 and Portuguese Patent Application No. 118181 filed Sep. 2, 2022.

### INCORPORATION BY REFERENCE OF SEQUENCE LISTING

The instant application contains a Sequence Listing which has been filed electronically in xml format and is hereby incorporated by reference in its entirety. Said xml copy, created on Mar. 4, 2024, is named 15302FFP.xml and is 4.8 KB in size.

### TECHNICAL FIELD OF THE INVENTION

The present invention relates to a method for synthesizing immunogenic chimeric substances comprising glycopeptides for use in generation of antibodies against cancer cells expressing glycopeptides and treatment and prevention of cancer by vaccination.

### STATE OF THE ART

Cancer cells often express at the surface abnormally O-glycosylated proteins showing immature simple mucin-type O-GalNAc glycans such as the Tn (GalNAcα-O-Ser/Thr) and sialyl-Tn (STn; Neu5Acα2-6GalNAcα-O-Ser/Thr) antigens, instead of more elongated and complex glycoforms.

These protein glycoforms may be generated by distinct mechanisms, such as mutations in enzymes involved in glycans biosynthesis, disorganization of secreting organelles as well as microenvironmental cues influencing sugars metabolism and glycogenes expression (1, 2). Their detection in solid tumours of different origins and etiologies is frequently associated with aggressiveness, resistance to different types of cancer therapies, and worst prognosis (3-6). Moreover, these glycans play a central role supporting multiple cancer hallmarks, including resistance to apoptosis, enhanced cell motility, invasion, metastasis, and immune escape (2). Therefore, several attempts have been made to explore them as cancer biomarkers and develop glycan-based targeted therapeutics, including monoclonal antibodies and CAR-Ts (7, 8). However, the mild expression of Tn and STn antigens across most organs of the gastrointestinal, respiratory tracts and the male and female reproductive systems challenges their cancer specificity and raises concerns for potentially deleterious off-target effects in therapeutic context. Their overexpression in non-malignant inflammatory conditions is also a major drawback for precision oncology, namely concerning cancer detection (9). The generalization of this approach has been delayed by the lack of identification of pan-carcinomic molecular signatures showing high cancer specificity.

### SUMMARY OF THE INVENTION

Due to its remarkable cancer specificity, disclosed herein are CD44s-Tn/STn glycopeptides for use in prophylactic and/or therapeutic cancer vaccines and ligands through precise targeting of aggressive cancer cells in primary tumours and metastasis as well as in peripheral blood circulation. Other uses comprise development of monoclonal antibodies for cancer detection and therapy. As example the 30-mer peptide with

SEQ ID NO: 1, carrying a motif specific to isoforms lacking the extracellular region encoded by variable exons 6-14, serves as a scaffold for one-pot glycosylation to yield a mixture of CD44s glycoproteoforms presenting one and/or more serine or threonine residues substituted with the Tn antigen (herein termed as CD44s-Tn). The said glycoproteoforms result from the glycosylation of the said 30-mer peptide by combination of multiple polypeptide N-acetylgalactosaminyltransferases (GalNAc-Ts), namely GalNAc-T1+GalNAc-T2+GalNAc-T3+GalNAc-T11 and UDP-GalNAc, in a single reaction. Purification of reaction products by VVA (Vicia Villosa) lectin affinity chromatography is introduced to remove residual amounts of the non-glycosylated original peptide. The process produces a mixture of multiple glycoproteoforms intended to reflect the micro, macro, and meta heterogeneity of CD44s glycosylation found in vivo, and providing vaccine multivalency (Table 1 and 2).

TABLE 1
CD44-Tn glycopeptides synthesized using different
combinations of polypeptide N-acetylgalactosaminyl
transferases, followed by VVA enrichment.
CD44s peptide sequence
DSPWITDSTDRIPATRDQDTFHPSGGSHTT

| | |
|---|---|
| # Glycosylated sites | Relative abundance (%) |

GalNAc-T1 + GalNAc-T2

| | |
|---|---|
| 1 Tn | 87 |
| 2 Tn | 11 |
| 3 Tn | 2 |

GalNAc-T1 + GalNAc-T3

| | |
|---|---|
| 1 Tn | 3 |
| 2 Tn | 33 |
| 3 Tn | 25 |
| 4 Tn | 31 |
| 5 Tn | 7 |

GalNAc-T2 + GalNAc-T3

| | |
|---|---|
| 1 Tn | 10 |
| 2 Tn | 33 |
| 3 Tn | 42 |
| 4 Tn | 13 |
| 5 Tn | 2 |

GalNAc-T1 + GalNAc-T2 + GalNAc-T3

| | |
|---|---|
| 1 Tn | 7 |
| 2 Tn | 35 |
| 3 Tn | 25 |
| 4 Tn | 27 |
| 5 Tn | 6 |

GalNAc-T1 + GalNAc-T3 + GalNAc-T11

| | |
|---|---|
| 1 Tn | 5 |
| 2 Tn | 38 |
| 3 Tn | 29 |
| 4 Tn | 21 |
| 5 Tn | 7 |
| 6 Tn | 1 |

GalNAc-T2 + GalNAc-T3 + GalNAc-T11

| | |
|---|---|
| 1 Tn | 10 |
| 2 Tn | 32 |
| 3 Tn | 34 |
| 4 Tn | 22 |
| 5 Tn | 3 |

GalNAc-T1 + GalNAc-T2 + GalNAc-T3 + GalNAc-T11

| | |
|---|---|
| 1 Tn | 5 |
| 2 Tn | 17 |
| 3 Tn | 36 |
| 4 Tn | 31 |
| 5 Tn | 11 |

TABLE 2
CD44-Tn/STn glycopeptides synthesized GalNAc-T1 plus GalNAc-T2
plus GalNAc-T3, followed by VVA enrichment and ST6GalNAc I.
CD44s peptide sequence
DSPWITDSTDRIPATRDQDTFHPSGGSHTT

| | |
|---|---|
| # Glycosylated sites | Relative abundance (%) |

GalNAc-T1 + GalNAc-T2 + GalNAc-T3 and ST6GalNAc I

| | |
|---|---|
| 1 Tn + 1 STn | 2 |
| 1 In + 2 STn | 5 |
| 1 Tn + 3 STn | 17 |
| 1 Tn + 4 STn | 7 |
| 2 Tn + 2 STn | 2 |
| 2 Tn + 3 STn | 1 |
| 1 STn | 2 |
| 2 STn | 31 |
| 3 STn | 26 |
| 4 STn | 6 |
| 5 STn | 1 |

In another embodiment, the same glycopeptides can be synthesized carrying a cysteine-tagged C-terminal motif, to be employed in the synthesis of glycovaccines chimeras with immunogens. A multivalent chimeric protein immunogen is synthetised to achieve more efficient delivery of the C-terminal cysteine-tagged CD44s-Tn glycoepitopes to antigen-presenting cells. The adopted strategy employs m-maleimidobenzoyl-N-hydoxysuccinimide ester (MBS) to covalently link immunogenic proteins such as CRM197, KLH, or any other glycoprotein of interest to CD44s-Tn glycopeptides, or any other glycopeptides of interest, carrying a C- or N-terminal cysteine tag. Conventionally, the synthesis consists firstly in the activation of the carrier protein with MBS, followed by incubation of the activated protein with the glycopeptide to achieve conjugation. The conventional approach may lead to protein-protein cross-links, originating multimerization and reduced linking to glycoepitopes. Protein carriers' multimerization may also generate macromolecular and potentially harmful clothing undesired compounds for vaccine applications. Taking this into account, a changed synthesis order, first activating the glycopeptide at 4° C. followed by incubation of the activated glycopeptide with the carrier protein, surprisingly overcomes this limitation. Multivalent glycochimeras comprehending KLH covalently linked via MBS to C-terminal cysteine-tagged CD44s-Tn glycopeptides (herein termed as KLH-CD44s-Tn) are to induce immune responses in human dendritic cells in vitro and in immunocompetent mice. The formulation does not show signs of toxicity in vitro and in vivo and induces increased IgM and IgG titers in mice, including the generation of anti-CD44s-Tn specific antibodies. Increased immune cell response and evidence of cellular memory is also observed, which is further by the administration of the adjuvant monophosphoryl-lipid A (MPLA).

### DETAILED DESCRIPTION

The present invention relates to glycopeptides characterized by, comprising a scaffold peptide sequence of the short CD44 isoforms resulting from alternative splicing of exons 6-14 (CD44s), with one or multiple residues selected from the list consisting of serine and threonine, substituted with antigens selected from the list consisting of Tn, STn and combinations thereof (CD44s-Tn/STn).

In one embodiment, the said scaffold peptide sequence is characterized by, comprising any peptide sequence within the said CD44s isoforms comprising the amino acid sequence motif consisting of SED ID NO:4).

In another embodiment, the said scaffold peptide sequence used to generate CD44s-Tn/STn glycopeptides is characterized by, comprising a peptide selected from the list consisting of: SED ID NO:1, SED ID NO:2, SEQ ID NO:3 and combinations thereof.

Another aspect of the present invention refers to methods of synthesis of the said CD44s-Tn/STn glycopeptides.

In one embodiment the method synthesizing the said CD44s-Tn/STn glycopeptides comprises the steps of:
a) glycosylation of the desired peptide chains by combining UDP-GalNAc with one or multiple polypeptide N-acetylgalactosaminyltransferases in 125 mM sodium cacodylate, 50 mM MnCl2 pH 7.4 buffer overnight at 37° C.
b) affinity purification of the CD44s-Tn/STn mixtures.

In one embodiment, the said polypeptide N-acetylgalactosaminyltransferases are characterized by, comprising GalNAc-T1, GalNAc-T2, GalNAc-T3, GalNAc-T11 and combinations thereof.

In another embodiment, the said affinity purification of the CD44-Tn/STn mixtures is characterized by, comprising affinity to agarose-bound Vicia Villosa Lectin (VVA).

In another embodiment, the said affinity purification of the CD44s-Tn/STn mixtures is characterized by, comprising the steps of:
a) affinity chromatography with agarose-bound VVA.
b) washing the column with 20 mM Tris-HCl pH 7.4, 150 mM NaCl, 1 mM CaCl2, MgCl2, MnCl2, and ZnCl2 buffer.
c) eluting bound glycopeptides in 3% acetic acid.
d) drying in a speed vac.

In another embodiment, the said affinity purification of the CD44s-Tn/STn mixtures is characterized by comprising the steps of:
a) resuspension in 50 mM MES, 20 mM EDTA, 2 mM DTT, pH 6.5.
b) incubation with ST6GalNAc I and GMP-Neu5Ac at 37° C. overnight.

In another embodiment, the said affinity purification of the CD44-Tn/STn mixtures is characterized by, a stationary phase separation of sialyated neutral glycopeptides, non-limiting examples of which comprise TiO2 affinity chromatography, C18 reverse phase liquid chromatography, hydrophilic interaction liquid chromatography (HILIC) and zwitterionic HILIC chromatography.

In another embodiment, the said CD44s-Tn/STn glycopeptides are characterized by, further comprising a conjugation to an immunogenic protein.

In one embodiment, the said CD44s-Tn/STn glycopeptides are characterized by, further comprising a cysteine-tag to enable covalent linkage of the glycopeptide N- or C-terminus to the said immunogenic protein.

In one embodiment, the said immunogenic protein is characterized by, comprising proteins capable of stimulating the immune system, non-limiting examples of which comprise keyhole limpet hemocyanin (KLH), cross-reacting material 197 (CRM197), tetanus toxoid and bovine serum albumin, and combinations thereof.

Another aspect of the present invention refers to compositions characterized by, comprising the said CD44s-Tn/STn glycopeptides conjugated to an immunogenic protein, as mentioned above.

In one embodiment, the said compositions are characterized by, comprising CD44s-Tn/STn glycopeptides conjugated to an immunogenic protein, in a polyvalent form.

In another embodiment, the said compositions are characterized by, further comprising other substances that protect the antigenic cargo and ensure their precise delivery, non-limiting examples of which comprise encapsulating liposomes, biocompatible polymeric substances, biocompatible polymeric polymers, nanoparticles, nanoparticles made of lipids, nanoparticles made of polysaccharides and combinations thereof.

In another embodiment, the said compositions are characterized by, further comprising other adjuvants that stimulate immune responses, selected from the group consisting of LTR 192G, aluminium hydroxide, RC529E, QS21, E294, oligodeoxynucleotides (ODN), CpG-containing oligodeoxynucleotides, aluminium phosphate and combinations thereof.

The CD44s-Tn glycopeptide is conjugated by click chemistry with immunogenic protein carriers CRM197 and KLH via MBS, an amine-to-sulfhydryl crosslinker that contains NHS-ester and maleimide reactive groups at opposite ends of a short aromatic spacer arm. The NHS-ester is intended to target primary amines in the peptide chain, whereas the maleimide group reacts with the thiol group at the CD44-Tn glycopeptides c-terminal. The objective is to produce CRM197-CD44s-Tn and KLH-CD44s-Tn chimeric glycopeptides. CRM197 is used to demonstrate that activation of the protein carrier with MBS induces significant multimerization by cross-linking (SDS-PAGE; FIG. 1A), even though it does not significantly affect the formation of CRM197-CD44s-Tn glycoconjugates (VVA western blot; FIG. 1A). To limit uncontrolled protein carrier cross-linking and avoid the formation of multimeric conjugates that may be deleterious for clinical applications, the reaction steps were inverted. The experimented method starts by activating CD44s-Tn glycopeptides with MBS at low temperature followed by incubation with the protein carrier. Surprisingly, according to the SDS-PAGE in FIG. 1B, this originates more homogeneous and lower molecular weight CRM197-CD44s-Tn chimeras, confirmed also by VVA western blots. This inverted conjugation method is also applicable to generate KLH-CD44s-Tn glycochimeras, as demonstrated by the slot blots in FIG. 2. The absence of VVA signals for controls with KLH, KLH-MBS, and CD44s confirms the specificity of VVA affinity for Tn antigens in the chimeras.

Through the above-mentioned experiments, it was possible to develop a method for preparation of CD44-Tn/STn glycopeptides linked to an immunogenic protein characterized by comprising the steps of:
a) activating CD44s-Tn/STn glycopeptides with an amino-to-sulfhydryl crosslinker MBS (m-maleimidobenzoyl-N-hydroxysuccinimide ester), at 4° C.
b) incubating with the immunogenic protein.
c) desalting and purification of the chimeric glycoconjugates in a PD-10 column.

In one embodiment, the said purification of the chimeric glycoconjugates is characterized by comprising a chromatographic method that enables the isolation of the said substances from other conjugation reagents and by-products, non-limiting examples of which comprise size exclusion chromatography, affinity chromatography for Tn and/or STn antigens and combinations thereof.

Human monocyte-derived dendritic cells isolated from healthy human donors are used to evaluate the immune cell response against the compositions as well as the composition's toxicity in vitro. All compositions (KLH, KLH-CD44s-Tn, KLH-CD44s-Tn plus MPLA) show neglectable signs of toxicity compared to the controls (FIG. 3). Moreover, an increase in CD86 and HLA-DR levels in dendritic cells treated with KLH and KLH-CD44s-Tn is observed compared to controls, even though not statistically significant due to the low number of replicas. In vivo, KLH-CD44s-Tn multivalent chimeric glycopeptides are administered to immunocompetent mice alone or co-adjuvanted with MPLA to boost immune responses. Mice are immunized three times with a week interval with PBS (sham control), KLH, KLH-CD44-Tn, and KLH-CD44-Tn plus MPLA. A week after the third immunization, mice are humanely euthanized and the organs (kidney, spleen, pancreas, and liver) collected to screen for signs of toxicity. Furthermore, spleen and lymph nodes are also harvested for cellular immune responses evaluation, whereas the blood is collected to access humoral responses, namely IgM and IgG titers and the existence of antibodies recognizing CD44s-Tn. Regarding composition's toxicity profile, during the immunization period, the weight of all animals increases over time in all groups administrated with immunogens (KLH; KLH-CD44s-Tn; KLH-CD44s-Tn+MPLA) and does not vary significantly from the control (FIG. 4). Furthermore, no mortality is observed. Histological analysis of several organs (spleen, liver, pancreas, kidney) do not show any alterations in comparison to the control group, including signs of necrosis, inflammation, or other toxicity induced morphological changes (FIG. 5). Collectively, these findings show that all formulations are well tolerated by the animals, demonstrating low/no toxicity.

In regard to humoral responses and specificity of antibody production, blood is collected and serum isolated from mice on week after the third immunization and IgM and IgG titers are accessed by ELISA. According to FIG. 6, both types of immunoglobulins are elevated in KLH, KLH-CD44s-Tn, and KLH-CD44s-Tn plus MPLA groups, being statistically significant for KLH-CD44s-Tn with and without MPLA administration. Furthermore, MPLA administration contributes to significantly increase IgG levels. IgMs and IgGs are then isolated from the serum of by protein L- and G-agarose chromatography, respectively. The presence of anti-CD44s-Tn specific antibody responses is accessed by dot blotting using non-glycosylated CD44s and KLH-CD44s chimeras and BSA as negative controls (FIG. 7). According to FIG. 7, all mice present IgM and IgGs against CD44s-Tn but not to the non-glycosylated CD44s as well as BSA, demonstrating the specificity of produced antibodies against the CD44s-Tn glycoimmunogens. In addition, some mice exhibited antibody responses (mostly IgMs) against KLH and KLH conjugates with both CD44s and CD44s-Tn glycopeptides.

In one embodiment the above-mentioned glycopeptides, glycopeptide conjugates and glycopeptide conjugate compositions are employed in generating antibodies characterized by, specifically recognizing native short CD44-Tn/STn glycoproteoforms, synthetic CD44s-Tn/STn glycopeptides, glycopeptide conjugates and combinations thereof.

It's possible to develop a method to produce the above-mentioned antibodies, characterized by comprising the steps of:
a) inoculation immunocompetent animals by one of different routes.
b) collecting blood from animals and then centrifuging at 2500 rpm for 30 min at RT.
c) collecting the serum fraction and again centrifuging at 1200 rpm for 5 min at 4° C.
d) affinity purification in a stationary phase bound to the said glycopeptides, glycopeptide conjugates or compositions thereof.
e) washing the stationary phase with 20 mM Tris-HCl pH 7.4, 150 mM NaCl, 1 mM CaCl2, MgCl2, MnCl2, and ZnCl2 buffer.

In another embodiment, the said affinity purification of the CD44s-Tn/STn antibodies is characterized by, comprising the steps of:
a) resuspension in 50 mM MES, 20 mM EDTA, 2 mM DTT, pH 6.5.
b) incubation with ST6GalNAc I and GMP-Neu5Ac at 37° C. overnight.

In another embodiment, the said affinity purification of the CD44s-Tn/STn antibodies is characterized by, a stationary phase separation, non-limiting examples of which comprise TiO2 affinity chromatography, C18 reverse phase liquid chromatography, hydrophilic interaction liquid chromatography (HILIC) and zwitterionic HILIC chromatography.

In other embodiments of the present method, the said antibodies may be generated by another methodology characterized by, comprising a step of:
a) presenting the said CD44s-Tn/STn glycoepitopes to the immune system leading to the generation of antibodies.

Another aspect of the present invention regards the uses of the said CD44s-Tn/STn antibodies.

Thus, another embodiment regards the said CD44s-Tn/STn glycopeptide-derived antibodies for use in detection of CD44s isoforms in tumours, circulating tumour cells, metastases, bodily fluids, extracellular vesicles and other cellular bodies.

Another embodiment regards the said CD44s-Tn/STn glycopeptide-derived antibodies for use in targeting cancer cells for delivery of therapeutic agents.

Another embodiment regards the said CD44s-Tn/STn glycopeptide-derived antibodies for use in the treatment of cancer, through targeting and induction of cancer cell's death.

Another embodiment regards the said CD44s-Tn/STn glycopeptide-derived antibodies for use in the treatment of cancer, through inducing immune responses against cancer cells.

Another embodiment regards the said CD44s-Tn/STn glycopeptide-derived antibodies for use in the treatment of cancer, through inducing a deleterious effect in cancer cells, promoting their elimination.

Regarding cellular immune response, immunization with KLH-CD44s-Tn, adjuvanted or not with MPLA, induced an immune response mediated by B-cells, CD4+, CD8+ T cells, and innate myeloid immune cells (including Type 1 Macrophages and dendritic cells; FIG. 8-9), together with an increased humoral response (FIG. 6). These mice also had a significant increment of the percentage of memory CD8+ and memory CD4+ T cells in the spleen and in the lymph nodes, suggesting long term memory to the administered antigens. This is consistent with the observed development of a humoral response against CD44s-Tn (FIGS. 6 and 7). Additionally, KLH-CD44s-Tn administration increased CD8+ cytotoxic T cells. In fact, mice immunized with the glycochimera experienced splenic B-cells clonal expansion, represented by the significant increase of the percentage of CD19 positive cells in the spleen. Furthermore, the spleen of chimera immunized mice had more activated B-cells (increase in MFI of MHC-II), resulting in differentiation into memory B-cells and plasma cells, contributing to the development of a humoral response. CD8+ T cells, B-cells and likely CD4+ T cells from the spleens of the chimera immunized mice proliferated with a specific antigen. Taken together, these observations reflect the potential of the KLH-CD44s-Tn glycovaccine in developing a specific humoral and cellular immune response against the CD44s-Tn cancer-associated signature.

Thus, another aspect of the present invention concerns glycopeptides, glycopeptide conjugates and compositions thereof as described above for use in a vaccine treatment for preneoplastic diseases and cancer therapy, for prevention of cancer development and for preventing or delaying relapse, through generating immunological responses and immunological memory against cancer cells, after administrating the said glycopeptides, conjugates and compositions thereof to humans or other animals.

Another embodiment regards glycopeptides, glycopeptide conjugates and compositions thereof as described above for use in a vaccine treatment for preneoplastic diseases and cancer therapy, for prevention of cancer development and for preventing or delaying relapse, administered orally, nasally, subcutaneously, intradermally, transdermally, transcutaneously, intramuscularly or rectally.

Another embodiment regards glycopeptides, glycopeptide conjugates and compositions thereof as described above for use in a vaccine treatment for pre-neoplastic diseases and cancer therapy, prevention of cancer development and for preventing or delaying relapse in combinations comprising other agents employed in prevention and treatment of primary tumours or disseminated disease, non-limiting examples of which comprise vaccines, anti-cancer chemotherapy drugs, immune check-point inhibitors, immunotherapies, radiotherapy and combinations thereof.

Another embodiment regards glycopeptides, glycopeptide conjugates and compositions thereof as described above for use in a vaccine treatment for pre-neoplastic diseases and cancer therapy, for prevention of development and for preventing or delaying relapse of pre-neoplastic lesions and cancers expressing CD44 short isoforms.

In conclusion, the following disclosure describes a CD44s-Tn/STn glycopeptides to be used as immunogens in vaccines targeting aggressive cancer cells, and the synthesis methods thereof. Methods for conjugating the synthesized CD44s-Tn glycopeptides to the immunogenic protein carriers CRM197 and KLH, are described, generating the chimeric glycopeptides, herein termed CRM197-CD44s-Tn and KLH-CD44s-Tn, respectively. It also shows the toxicity of the KLH-CD44s-Tn formulations in vitro against human dendritic cells in vitro and in vivo in immunocompetent mice. Finally, it evaluates the nature of humoral and cellular immune responses elicited by KLH-CD44s-Tn in vivo, alone or co-adjuvanted with MPLA.

### BRIEF DESCRIPTION OF THE FIGURES

FIG. 1: Reaction products of CRM197-MBS-CD44s-Tn conjugation by SDS-PAGE and VVA lectin western blotting. A) Conjugation involving CRM197 activation with MBS followed by the addition of CD44s-Tn. The SDS-PAGE for CRM197 shows a major band at 50 kDa (left lane). In the presence of MBS before (middle lane) and after incubation with CD44s-Tn glycopeptides (right lane) the gels presented poorly defined smears spanning above 50 to above 250 kDa) supporting the existence of multiple cross-links in the protein carrier. Corresponding VVA lectin blots showed no Tn antigens in both CRM197 and activated CRM197 but confirmed the presence of these glycans in the CRM197-CD44s-Tn chimera. B) Conjugation involving CD44s-Tn activation with MBS followed by the addition of CRM197. Activation of CD44s-Tn glycopeptides with MBS prior to incubation with CRM197 generated more homogeneous synthesis products characterized by only two very well-defined bands above 50 and close to 150 kDa. VVA blotting confirmed the successful conjugation of CD44s-Tn glycopeptides.

For western-blot the products of the CRM197 conjugation for both strategies were resolved by SDS-PAGE on 4%-20% precast polyacrylamide gels and stained with Coomasie Brilliant Blue G-250. In addition, the conjugated glycopeptides were also transferred onto 0.45 μm nitrocellulose membranes for further STn and Tn evaluation by western blot. Briefly, membranes were blocked with Carbo-free blocking solution and blotted with biotinylated VVA lectin (Tn; 1:5000) or biotinylated SNA lectin (STn; 1:5000) for 1 hour at RT and then incubated with the Vectastain Elite ABC reagent, Peroxidase, R.T.U.

FIG. 2: Validation of KLH-CD44s-Tn synthesized by the MBS-activated glycopeptides approach by dot blotting. KLH, KLH-MBS, and CD44s showed no reactivity to the VVA lectin. On the other hand, dots were observed in the area corresponding to CD44s-Tn, MBS-CD44s-Tn, and KLH-CD44s-Tn glycoconjugates.

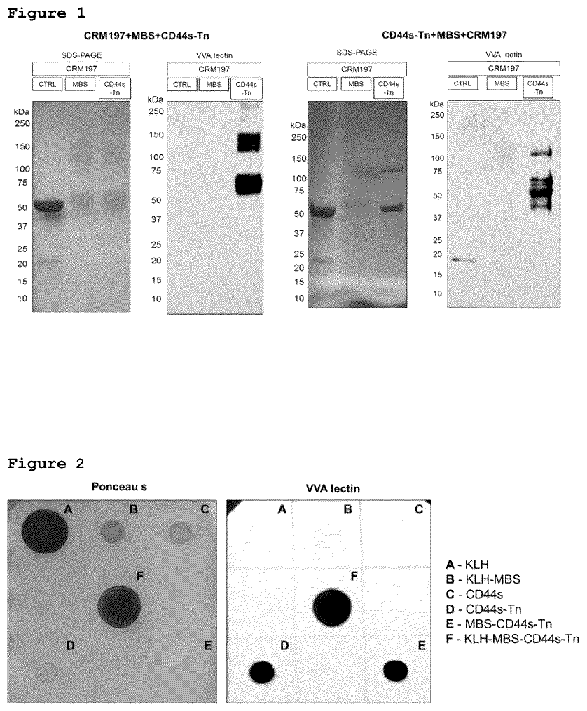

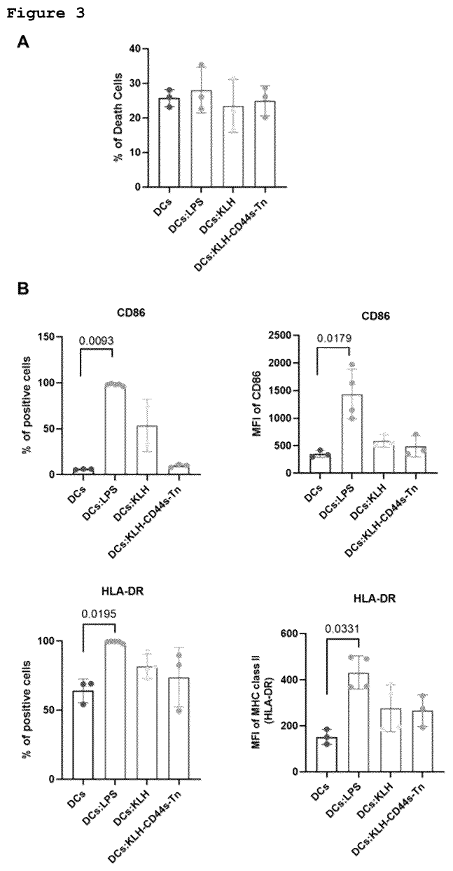

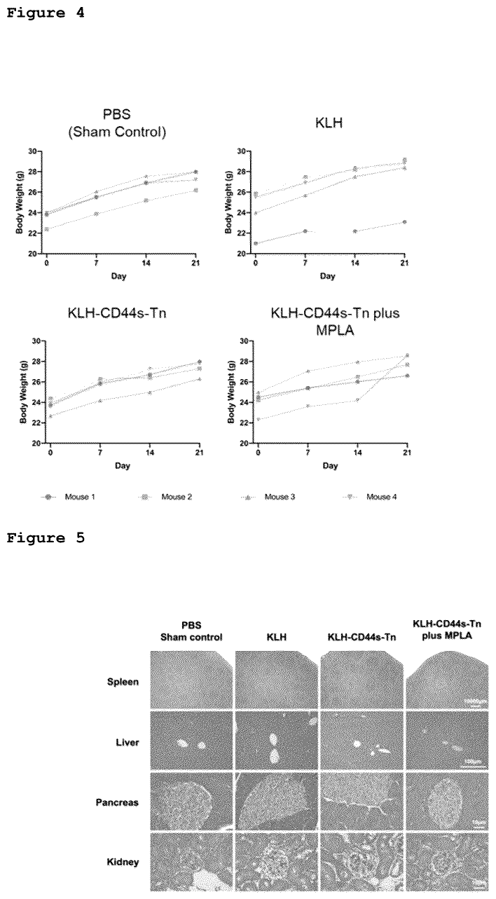

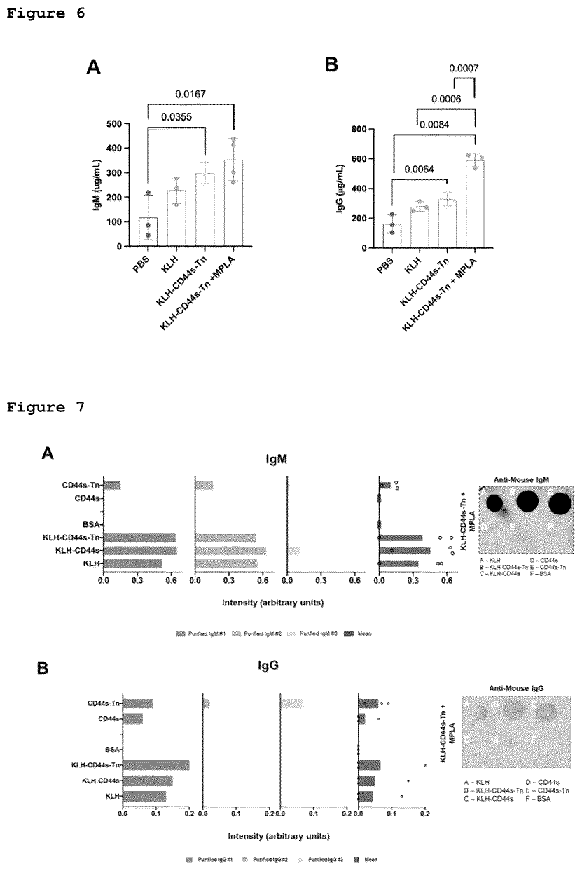

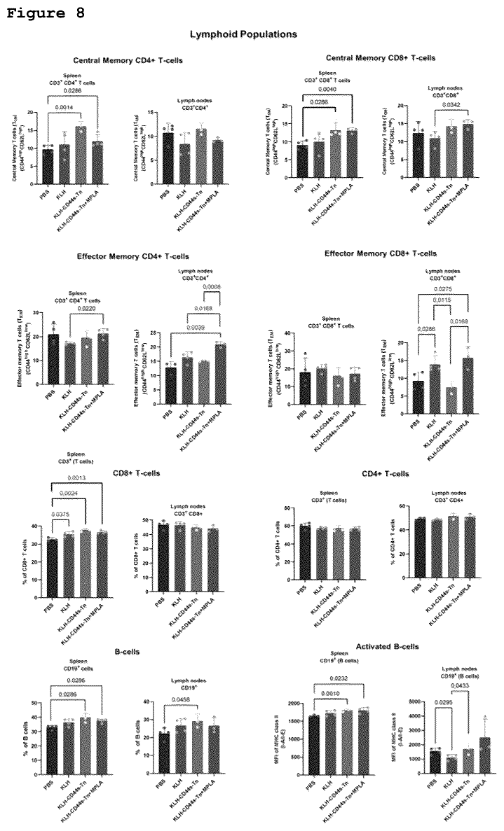

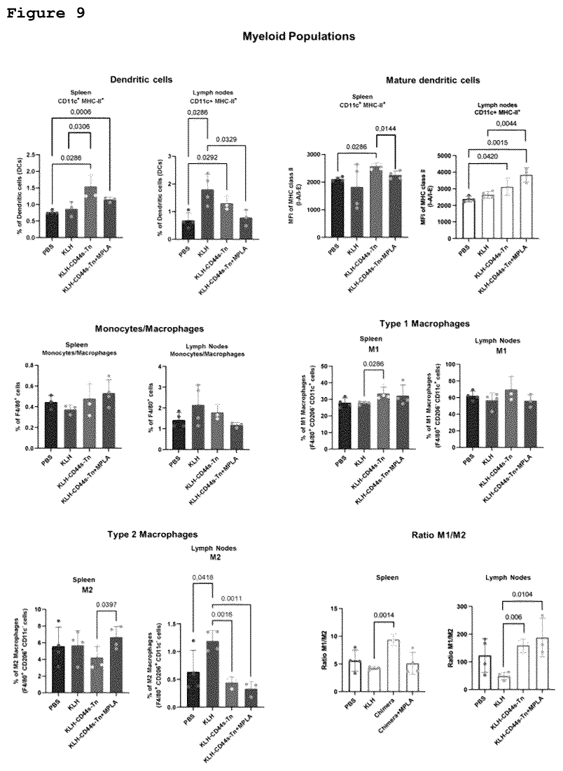

For dot blotting, the products of the KLH conjugation were analysed by dot blotting. Nitrocellulose membranes (0.45 μM) were loaded with 10 μg of KLH, KLH-MBS, KLH-CD44s-Tn, KLH-CD44s, CD44s, CD44s-Tn, and MBS-CD44s-Tn. The membranes, previously blocked with Carbo-free blocking solution, were blotted for the Tn antigen with biotinylated VVA lectin (1:5000) for 1 hour at RT and finally incubated with Vectastain Elite ABC reagent, Peroxidase, R.T.U. The Amersham ECL Prime Western Blotting Detection Reagent was used as developing reagent. Data analysis was performed through Image Lab Software in a ChemiDoc XRS (Bio-Rad).

FIG. 3: Evaluation of KLH-CD44s-Tn glycochimeric vaccines toxicity and activation of monocyte-derived dendritic cells in vitro. A) KLH-CD44s-Tn are non-toxic to human dendritic cells in vitro. KLH-CD44s-Tn glycochimeras do not induce significant cell death compared to unstimulated (DCs), and LPS and KLH-activated dendritic cells. B) KLH-CD44s-Tn do not significantly activate dendritic cells in vitro. KLH-CD44s-Tn glycochimeras do not induce significant increase in CD86 and HLA-DR in comparison to unstimulated (DCs), and LPS activated dendritic cells. However, KLH-CD44s-Tn stimulated DCs showed a trend increase for both markers in comparison to unstimulated DCs, suggesting potential activation.

Human monocytes were isolated from buffy coats from healthy blood donors. Buffy coats were obtained from Hospital São João (Porto, Portugal). Briefly, peripheral blood mononuclear cells (PBMCs) were collected from previously centrifuged buffy coats (30 min, 1200×g, RT, without brake), and incubated with RosetteSep human monocyte enrichment kit, according to manufacturer's instructions. This mixture was then diluted 1:1 in PBS supplemented with 2% FBS, layered over Histopaque-1077, and centrifuged as previously. The enriched monocyte layer was collected and washed three times with PBS. For monocyte-DC differentiation, 1×106 monocytes were seeded on 6-well plates and differentiated for 5 days in complete RPMI-1640 medium (supplemented with 10% FBS and 1% Pen Strep), supplemented with 50 ng/mL IL-4 and GM-CSF.

For dendritic cell-based in vitro assays for vaccine immunogenicity, the human monocyte-derived dendritic cells were cultured during 24 h with the formulations. 50 ng/mL LPS (Sigma) was used as DCs activation control. After 24 h of co-culture, conditioned mediums were collected and the adhered DCs were detached with PBS 1× on ice for 30 minutes. After centrifugation (5 min, 1500×g, 4° C.) DCs were resuspended in FACS buffer (PBS, 0.5-1% BSA or 5-10% FBS, 0.1% NaN3 sodium azide) and immune stained for flow cytometry analysis. mo-DCS differentiation and maturation states were assessed by immunostaining with FITC-anti-CD86 and APC-anti-CD11c at a dilution 1:25 in FACS buffer, and PE-anti-HLA-DR at a dilution 1:100 in FACS buffer. Cell viability was assessed through DAPI staining (750 ng/mL).

FIG. 4: Mice body weight (grams) variation in the four groups of immunization across the experimental timeline. Body weight was assessed using a digital scale immediately before each subcutaneous injection of the vaccine formulations (t=7, 14, and 21 days). An increase in body weight was observed for all animals enrolled in the experiment, suggesting that mice tolerate the administrated compounds.

Seven-week-old male C57BL/6 mice were used for this study. A total of 16 mice were acclimatized in the quarantine room at the animal facility for 1 week before entering the protocol. During this period, animals were monitored for any signs of distress that could compromise their suitability for experimental use. Mice were housed in groups of 4 per cage in a Makrolon type I cage in a limited access area at a controlled room temperature, 12 h day 12 h dark cycles, with food and water ad libitum. Environmental enrichment was provided, consisting of corn cob bedding, nesting material, tunnel, and chewing blocks.

For the immunization study, mice were randomly and blindly distributed into 4 groups (n=4 per group), namely Sham Control, KLH, KLH-CD44s-Tn, and KLH-CD44s-Tn-MPLA. Mice immunizations were performed subcutaneously in the dorsal region with each correspondent formulation, i.e., 100 μL of PBS (sham control group), 50 μg of KLH, 50 μg of KLH-CD44s-Tn or 50 μg of KLH-CD44s-Tn plus 20 μg MPLA adjuvant (100 μL). Each mouse was immunized three times at a one-week interval between them; the first immunization occurred on day 0, and the second and third occurred on days 7 and 14, respectively. One week following the last immunization, mice were humanely euthanized and blood and tissues (lymph nodes, pancreas, liver, kidney, and spleen) were collected for evaluation by histology, flow cytometry, western and dot blotting, and ELISA. Mice body weight change was monitored once per week, immediately before the immunizations. Blood was collected with heparin from all animals before and after immunizations by the tail vein and cardiac puncture at sacrifice time, respectively, and then centrifuged at 2500 rpm for 30 min at RT. The serum fraction was collected and again centrifuged at 1200 rpm for 5 min at 4° C., and finally stored at −80° C. for further use.

FIG. 5: Histological evaluation of mice splenic, hepatic, pancreatic, and renal tissue following immunization to different vaccine formulations. No significant histological alterations (necrosis, inflammation, additional toxicity-induced morphological alterations) were observed in the spleen, liver, pancreas, and kidney among the experimental groups.

The liver, spleen, kidney, and pancreas were collected to assess toxicity induced by the immunization with the different vaccine formulations. All fresh tissue samples were fixed and preserved using a 10% buffered formaldehyde solution (formalin) overnight. After fixation, tissue specimens were manually dehydrated using a graded ethanol series and xylene, and finally impregnated in paraffin at 60° C. (1 hour in each step, twice). Then, impregnated tissues were embedded in paraffin using a paraffin embedding system. Formalin-fixed paraffin-embedded (FFPE) tissue samples were cut in 3 μm-thick sections on a microtome with a disposable blade and transferred onto specific glass slides to perform H&E (haematoxylin and eosin) staining. Briefly, 3 μm FFPE tissue sections were deparaffinized and rehydrated, incubated for 2 and 5 min with eosin and haematoxylin, respectively, followed by quick washing in distilled water. After drying at RT, slides were submerged in xylene and mounted on Entellan. Microscopical analysis was performed to detect histological variations among groups, including necrosis, inflammation, fibrosis, anisokaryosis, and additional pathognomonic findings associated with toxicity.

FIG. 6: IgM and IgG titers one week after administration of the different formulations to immunocompetent mice. IgM and IgG titers increased in all groups containing the protein carrier in relation to the sham control (PBS), being statistically significant for KLH-CD44s-Tn and KLH-CD44s-Tn plus MPLA groups. Also, co-adjuvant MPLA significantly increases IgG titers in comparison to KLH-CD44s-Tn. Statistical analyses were conducted using a nonparametric Mann-Whitney test.

For antibody quantification, IgG and IgM antibodies concentration in serum samples was determined using Mouse IgG/IgM ELISA Antibody Pair Kit assay (STEMCELL Technologies) according to the manufacturer's instructions. Briefly, ELISA plates were coated with Mouse IgM or IgG capture antibody overnight at 4° C. After several washes, sera (IgM dilution: 1:100,000; IgG dilution: 1:50,000) were added, followed by incubation with the alkaline phosphatase-conjugated detection antibody (1:1000). At last, ELISA plates were washed and pNPP substrate was used to detect alkaline phosphatase. Absorbances were read at 405 nm using the iMARK microplate reader. All samples were analysed in duplicates.

FIG. 7: Evaluation of anti-CD44s-Tn IgMs and IgGs in the serum of KLH-CD44s-Tn plus MPLA immunized mice in comparison to the sham control (PBS group). IgMs (A) and IgGs (B) isolated from mice inoculated with PBS (sham control) and KLH-CD44s-Tn plus MPLA were screened for anti-CD44s-Tn antibodies against CD44s-Tn glycopeptides immobilized in nitrocellulose membranes. CD44s peptides, BSA, KLH-CD44s-Tn, KLH-CD44s, and KLH were used as controls. All mice produced anti-CD44s-Tn IgMs and IgGs with minor or no cross-reactivity with CD44s. Antibodies against KLH as well as KLH-expressing chimeras were observed for some mice, mostly of the IgM subtype. No anti-BSA antibodies were detected, supporting antibody specificity.

For IgG and IgM antibodies isolation, sera IgGs and IgMs were isolated with protein G-agarose and protein L-agarose beads, respectively. Initially, agarose beads were washed with PBS (pH 7.4) and then sera were incubated with beads for 3 h at 4° C. under agitation. Thereafter, beads were washed with PBS to remove the non-coupled antibodies, and the antibodies were recovered in 0.2 M glycine (pH 2.0) solution. To avoid antibody denaturation, the solution was immediately exchanged by PBS using amicon ultra centrifugal 10 kDa filters.

For IgG and IgM affinity characterization, the affinity of IgG and IgM antibodies elicited by the immunization of the KLH-CD44s-Tn glycoconjugates was evaluated by dot blotting. Briefly, 10 μg of KLH, KLH-CD44s-Tn, KLH-CD44s, CD44s, CD44s-Tn, and BSA were loaded onto 0.45 μM nitrocellulose membranes. Then, membranes were blocked with Carbo-free blocking solution and blotted with the isolated IgG and IgM antibodies (1 μg/mL; 1 hour, RT) from sera of mice immunized with KLH-CD44s-Tn+MPLA glycoformulation. At last, Goat anti-mouse IgG secondary antibody HRP (1:40000) and Goat anti-mouse IgM secondary antibody HRP (1:1000) were incubated for 30 min at RT. Serum derived from the Sham control group (PBS administration) was used as control. The Amersham ECL Prime Western Blotting Detection Reagent was used as developing reagent. Data acquire and analysis were performed through Image Lab Software in a ChemiDoc XRS (Bio-Rad).

FIG. 8: Flow cytometric analysis of the main mouse lymphoid populations present in the spleen and lymph nodes of immunized and control C57BL/6 mice. Percentage of Central Memory CD4+ and CD8+ T cells (CD44highCD62Lhigh). Percentage of Effector Memory CD4+ and CD8+ T cells (CD44highCD62Llow). Percentage of CD8+ T cells and CD4+ T cells (CD45+ CD3+). Percentage of B-cells (CD45+ CD19+) and mean fluorescence intensity (MFI) of MHC-II of activated B cells. Data are presented as mean±SEM (n=4). Unpaired T-test was used for selected groups.

To produce lymph nodes and spleen single cell suspensions, the dorsal lymph nodes and spleens were collected and conserved in MACS tissue storage solution, and later mechanically macerated in PBS and filtered using a 70 μm cell strainer. The cell suspensions were centrifuged at 300×g/7 min/4° C., and then incubated with Red Blood Cells Lysis Buffer for 15 min at RT, and further centrifuged at 300×g/7 min/4° C. to remove the erythrocytes. Finally, lymph nodes and spleen cells were resuspended in 500 μL of FACS buffer (PBS, 2% FBS, 0.01% sodium azide), and cell viability and counting were assessed by trypan blue dye exclusion test using EVE Automated Cell Counter (NanoEnTek).

FIG. 9: Flow cytometric analysis of the mouse myeloid populations present in the spleen and the lymph nodes of immunized and control C57BL/6 mice. Percentage of dendritic cells (CD45+F4/80−CD11c+MHC-II+) and the mean fluorescence intensity (MFI) of MHC-II of mature dendritic cells. Percentage of Type 1 macrophages (M1) (F4/80+CD206−CD11c+) and Type 2 macrophages (M2) (F4/80+CD206+CD11c−). Ratio M1/M2 macrophages. Data are presented as mean±SEM (n=4). Unpaired T-test was used for selected groups. For flow cytometry, the lymph nodes and spleen cells were stained with two different monoclonal antibody panels and analysed by flow cytometry in order to characterize different immune cell populations (T, B and dendritic cells, macrophages/monocytes and neutrophils). Approximately 1 million cells were washed in FACS buffer and blocked with Fc blocking agent for 15 min, to minimise non-specific antibody binding. Then, cells were stained in the dark, for 30 min at RT, with the following antibodies: CD19-PB, CD3-PO, CD4-APC-H7, CD8-PE-Cy7, CD44-PB, CD62L-FITC, CD45-PerCP5.5, CD206-APC, Ly6G-APC-H7, F4/80-FITC, and I-A/I-E-PE. After washing with FACS buffer, cells were acquired on a NAVIOS Flow Cytometer (Beckman Coulter). The FACS data were analysed using the Infinicyt software (version 1.7, Cytognos SL, Salamanca, Spain) to discriminate cell populations and determine the mean fluorescence intensity (MFI), and the percentage of positive cells (% positive cells).

### EXAMPLES

### Example 1—Generation of CD44s-Tn Glycopeptides From a C-Terminal Cysteine-Tagged Peptide Derived From CD44s

Peptide synthesis is performed chemically either in solution or on a solid phase. The process involves directed and selective formation of an amide bond between an N-protected amino acid and an amino acid bearing a free amino group and protected carboxylic acid. In solid phase synthesis, the carboxyl protecting group is linked to a polymer support. Following bond formation, the amino-protecting group of the dipeptide is removed, and the next N-protected amino-acid is coupled. Synthetic peptides can be produced with the designated sequence.

A peptide derived from CD44s, which is cysteine-tagged at the c-terminal and presents the following amino acid sequence CDSPWITDSTDRIPATRDQDTFHPSGGSHTT, is used as scaffold for the generation of CD44s-Tn glycopeptides.

The peptide was solubilized in 125 mM sodium cacodylate, 50 mM MnCl2 (pH 7.4) together with UDP-GalNAc and human GalNAc-T1+GalNAc-T2+GalNAc-T3+GalNAc-T11. This combination was incubated at 37° C. under mild stirring for 12 h to generate the CD44-Tn glycopeptides. The synthesis products were purified by agarose-bound VVA-lectin by washing the column with 20 mM Tris-HCl pH 7.4, 150 mM NaCl, 1 mM CaCl2, MgCl2, MnCl2, and ZnCl2. The CD44-Tn glyocopeptides were eluted in 3% acetic acid. Purity assessment and glycopeptides characterization was conducted by C18 reverse phase nanoLC-HCD/CID-MS/MS. Analysis by nanoLC-MS/MS demonstrated the products described in Table 1.

TABLE 1
C-terminal cysteine-tagged CD44s-In glycopeptides synthesized by the
combination of multiple polypeptide N-acetylgalactosaminyl transferases
(GalNAc-T1, T2, T3, T11) followed by VVA lectin enrichment.
C-terminal cysteine-tagged CD44s peptide sequence
CDSPWITDSTDRIPATRDQDTFHPSGGSHTT

| | |
|---|---|
| # Glycosylated sites | Relative abundance (%) |

GalNAc-T1 + GalNAc-T2 + GalNAc-T3 + GalNAc-T11

| | |
|---|---|
| 1 Tn | 4 |
| 2 Tn | 17 |
| 3 Tn | 37 |
| 4 Tn | 31 |
| 5 Tn | 11 |

### Example 2—Conjugation Protocol 1 to Link the Cysteine-Tagged CD44s-Tn Glycopeptides to CRM197 or KLH

In one approach 0.01 M CRM197 or 0.01 mg/μL KLH in PBS was incubated for 30 minutes (min) at room temperature (RT) under agitation with 0.015 mg/μL MBS in dimethylformamide (DMF). Then, CRM197-MBS or KLH-MBS solutions were passed through a PD-10 desalting column conditioned with 0.05 M phosphate buffer (pH 6). To allow the mild reduction of the C-terminal cysteines, tris(2-carboxyethyl) phosphine (TCEP) was added to 10 μg glycopeptides at a final concentration of 3 μM and incubated for 1 hour at 4° C. with agitation. Subsequently, 10 μg of desalted CRM197-MBS or KLH-MBS solutions were added to 10 μg of reduced cysteine-tagged CD44s-Tn glycopeptides in 25 μL DMF pH 7.0 allowing the conjugation overnight at 4° C. under agitation. Finally, the conjugated glycopetides were desalted with PD-10 desalting columns to remove the uncoupled glycopeptides, and further concentrated in a 10 kDa amicon ultra-0.5 mL centrifugal filter.

### Example 3—Conjugation Protocol 2 to Link the Cysteine-Tagged CD44s-Tn Glycopeptides to CRM197 or KLH

A second approach starts with the mild reduction of the C-terminal cysteines by adding TCEP to 20 μg glycopeptides at a final concentration of 3 μM for 1-2 hours at 4° C. with agitation. Then, the reduced glycopeptides were incubated overnight at 4° C. under agitation with 10 μL of MBS (0.015 mg/μL) in DMF, and the resulting CD44s-Tn-MBS peptides were posteriorly passed through a 3 kDa Amicon to remove the non-functionalized fraction. At last, 20 μg of reduced cysteine-tagged CD44s-Tn-MBS glycopeptides were conjugated with 50 μg KLH or CRM197 (0.01 mg/μL) in PBS for 30 min at RT under agitation. The final conjugation products were further passed through a 50 kDa amicon for desalting and removing the uncoupled glycopeptides.

| | SEQUENCE LISTING |
|---|---|
| SEQ ID NO: 1 | |
| | DSPWITDSTDRIPATRDQDTFHPSGGSHTT |
| SEQ ID NO: 2 | |
| | STVHPIPDEDSPWITDSTDRIPATRDQDTF |
| SEQ ID NO: 3 | |
| | ITDSTDRIPATRDQDTF |
| SEQ ID NO: 4 | |
| | ATR |

### REFERENCES

1. Azevedo R, Peixoto A, Gaiteiro C, Fernandes E, Neves M, Lima L, et al. Over forty years of bladder cancer glycobiology: Where do glycans stand facing precision oncology? Oncotarget. 2017; 8(53):91734-64.
2. Peixoto A, Relvas-Santos M, Azevedo R, Santos LL, Ferreira JA. Protein Glycosylation and Tumor Microenvironment Alterations Driving Cancer Hallmarks. Front Oncol. 2019; 9:380.
3. Ferreira JA, Videira PA, Lima L, Pereira S, Silva M, Carrascal M, et al. Overexpression of tumour-associated carbohydrate antigen sialyl-Tn in advanced bladder tumours. Mol Oncol. 2013; 7(3):719-31.
4. Costa C, Pereira S, Lima L, Peixoto A, Fernandes E, Neves D, et al. Abnormal Protein Glycosylation and Activated PI3K/Akt/mTOR Pathway: Role in Bladder Cancer Prognosis and Targeted Therapeutics. PLoS One. 2015; 10(11):e0141253.
5. Santos SN, Junqueira MS, Francisco G, Vilanova M, Magalhaes A, Dias Baruffi M, et al. O-glycan sialylation alters galectin-3 subcellular localization and decreases chemotherapy sensitivity in gastric cancer. Oncotarget. 2016; 7(50):83570-87.
6. Mereiter S, Balmana M, Gomes J, Magalhaes A, Reis CA. Glycomic Approaches for the Discovery of Targets in Gastrointestinal Cancer. Front Oncol. 2016; 6:55.
7. Loureiro LR, Sousa DP, Ferreira D, Chai W, Lima L, Pereira C, et al. Novel monoclonal antibody L2A5 specifically targeting sialyl-Tn and short glycans terminated by alpha-2-6 sialic acids. Sci Rep. 2018; 8(1):12196.
8. Posey AD, Jr., Schwab RD, Boesteanu AC, Steentoft C, Mandel U, Engels B, et al. Engineered CAR T Cells Targeting the Cancer-Associated Tn-Glycoform of the Membrane Mucin MUC1 Control Adenocarcinoma. Immunity. 2016; 44(6):1444-54.
9. Gomes C, Almeida A, Ferreira JA, Silva L, Santos-Sousa H, Pinto-de-Sousa J, et al. Glycoproteomic analysis of serum from patients with gastric precancerous lesions. J Proteome Res. 2013; 12(3):1454-66.

Lisbon, 5^{th} September 2022

1. Glycopeptides characterized by, comprising a scaffold peptide sequence of the short CD44 isoforms resulting from alternative splicing of exons 6-14 (CD44s), with one or multiple residues selected from the list consisting of serine and threonine, substituted with antigens selected from the list consisting of Tn, STn and combinations thereof (CD44s-Tn/STn).

2. Glycopeptides according to claim 1, the said scaffold peptide sequence characterized by, comprising any peptide sequence within the said CD44s isoforms comprising the amino acid sequence motif consisting of SED ID NO:4).

3. Glycopeptides according to claim 1, the said scaffold peptide sequence characterized by, comprising a peptide selected from the list consisting of: SED ID NO:1, SED ID NO:2, SEQ ID NO:3 and combinations thereof.

4. Method of synthesis of the CD44s-Tn/STn glycopeptides described in claim 1 comprising the steps of:
a) glycosylation of the desired peptide chains by combining UDP-GalNAc with one or multiple polypeptide N-acetylgalactosaminyltransferases in 125 mM sodium cacodylate, 50 mM MnCl2 pH 7.4 buffer overnight at 37° C.;
b) affinity purification of the CD44s-Tn/STn mixtures.

5. Method according to claim 4, the said polypeptide N-acetylgalactosaminyltransferases characterized by, comprising GalNAc-T1, GalNAc-T2, GalNAc-T3, GalNAc-T11 and combinations thereof.)

6. Method according to claim 4, the said affinity purification of the CD44-Tn/STn mixtures is characterized by, comprising affinity to agarose-bound Vicia Villosa Lectin (VVA).

7. Method according to claim 4, the said affinity purification of the CD44s-Tn/STn mixtures characterized by, comprising the steps of:
a) affinity chromatography with agarose-bound VVA;
b) washing the column with 20 mM Tris-HCl pH 7.4, 150 mM NaCl, 1 mM CaCl2, MgCl2, MnCl2, and ZnCl2 buffer;
c) eluting bound glycopeptides in 3% acetic acid;
d) drying in a speed vac.

8. Method according to claim 4, the said affinity purification of the CD44s-Tn/STn mixtures characterized by, comprising the steps of:
a) resuspension in 50 mM MES, 20 mM EDTA, 2 mM DTT, pH 6.5;
b) incubation with ST6GalNAc I and GMP-Neu5Ac at 37° C. overnight.

9. Method according to claim 4, the said affinity purification of the CD44-Tn/STn mixtures is characterized by, a stationary phase separation of sialyated neutral glycopeptides.

10. Glycopeptides according to claim 4 characterized by, further comprising a conjugation to an immunogenic protein.

11. Glycopeptides according to claim 10 characterized by, further comprising a cysteine-tag to enable covalent linkage of the glycopeptide N- or C-terminus to the said immunogenic protein.

12. Glycopeptides according to claim 11 characterized by, comprising proteins capable of stimulating the immune system.

13. Method for preparation of CD44-Tn/STn glycopeptides linked to an immunogenic protein as described in claim 10 characterized by, comprising the steps of:
a) activating CD44s-Tn/STn glycopeptides with an amino-to-sulfhydryl crosslinker at 4° C.;
b) incubating with the immunogenic protein;
c) desalting and purification of the chimeric glycoconjugates in a PD-10 column.

14. Method according to claim 13, the said immunogenic protein is characterized by, comprising proteins capable of stimulating the immune system.

15. Method according to claim 13, the said purification of the chimeric glycoconjugates is characterized by comprising a chromatographic method that enables the isolation of the said substances from other conjugation reagents and by-products.

16. Pharmaceutical composition characterized by, comprising the said glycopeptides described in claim 1.

17. Compositions according to claim 16 characterized by, comprising CD44s-Tn/STn glycopeptides conjugated to an immunogenic protein, in a polyvalent form.

18. Compositions according to claim 16 characterized by, further comprising other substances that protect the antigenic cargo and ensure their precise delivery.

19. Compositions according to claim 16 characterized by, further comprising other adjuvants that stimulate immune responses, selected from the group consisting of LTR 192G, aluminum hydroxide, RC529E, QS21, E294, oligodeoxynucleotides (ODN), CpG-containing oligodeoxynucleotides, aluminum phosphate and combinations thereof.

20. Antibodies derived from the glycopeptides described in claim 1 characterized by, specifically recognizing native short CD44-Tn/STn glycoproteoforms, synthetic CD44s-Tn/STn glycopeptides, glycopeptide conjugates and combinations thereof.

21. Method to produce the above-mentioned antibodies, characterized by comprising the steps of:
a) inoculation immunocompetent animals by one of different routes;
b) collecting blood from animals and then centrifuging at 2500 rpm for 30 min at RT;
c) collecting the serum fraction and again centrifuging at 1200 rpm for 5 min at 4° C.;
d) affinity purification in a stationary phase bound to the glycopeptides described in claim 1;
e) washing the stationary phase with 20 mM Tris-HCl pH 7.4, 150 mM NaCl, 1 mM CaCl2, MgCl2, MnCl2, and ZnCl2 buffer.

22. Method according to claim 21, the said affinity purification of the CD44s-Tn/STn antibodies is characterized by, comprising the steps of:
a) resuspension in 50 mM MES, 20 mM EDTA, 2 mM DTT, pH 6.5;
b) incubation with ST6GalNAc I and GMP-Neu5Ac at 37° C. overnight.

23. Method according to claim 21, the said affinity purification of the CD44s-Tn/STn antibodies is characterized by, a stationary phase separation.

24. Method according to claim 21, the said antibodies may be generated by another methodology characterized by, comprising a step of:
a) presenting the said CD44s-Tn/STn glycoepitopes to the immune system leading to the generation of antibodies.

25. Antibodies as described in claim 20 for use in detection of CD44s isoforms in tumours, circulating tumour cells, metastases, bodily fluids, extracellular vesicles and other cellular bodies.

26. Antibodies as described in claim 20 for use in targeting cancer cells for delivery of therapeutic agents.

27. Antibodies as described in claim 21 for use in the treatment of cancer, through targeting and induction of cancer cell's death.

28. Antibodies as described in claim 20 for use in the treatment of cancer, through inducing immune responses against cancer cells.

29. Antibodies as described in claim 20 for use in the treatment of cancer, through inducing a deleterious effect in cancer cells, promoting their elimination.

30. Glycopeptides described in claim 1 for use in a vaccine treatment for preneoplastic diseases and cancer therapy, for prevention of cancer development and for preventing or delaying relapse, through generating immunological responses and immunological memory against cancer cells, after administrating the said glycopeptides, conjugates and compositions thereof to humans or other animals.

31. Glycopeptides described in claim 1 for use in a vaccine treatment for preneoplastic diseases and cancer therapy, for prevention of cancer development and for preventing or delaying relapse, administered orally, nasally, subcutaneously, intradermally, transdermally, transcutaneously, intramuscularly or rectally.

32. Glycopeptides described in claim 1 for use in a vaccine treatment for pre-neoplastic diseases and cancer therapy, prevention of cancer development and for preventing or delaying relapse in combinations comprising other agents employed in prevention and treatment of primary tumours or disseminated disease.

33. Glycopeptides described in claim 1 for use in a vaccine treatment for pre-neoplastic diseases and cancer therapy, for prevention of development and for preventing or delaying relapse of pre-neoplastic lesions and cancers expressing CD44 short isoforms.


SEQUENCE LISTING (4 sequences)

SEQ ID NO: 1 - protein, 30 aa, Homo sapiens
DSPWITDSTDRIPATRDQDTFHPSGGSHTT

SEQ ID NO: 2 - protein, 30 aa, Homo sapiens
STVHPIPDEDSPWITDSTDRIPATRDQDTF

SEQ ID NO: 3 - protein, 17 aa, Homo sapiens
ITDSTDRIPATRDQDTF

SEQ ID NO: 4 - protein, 7 aa, Homo sapiens
SEQIDNO